(19) United States
(12) Patent Application Publication
(10) Pub. No.: US 2008/0009843 A1
(43) Pub. Date: Jan. 10, 2008

(54) SURGICAL ABLATION SYSTEM WITH CHEST WALL PLATFORM

(76) Inventor: Ralph de la Torre, Newton, MA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/761,563

(22) Filed: Jun. 12, 2007

Related U.S. Application Data
(60) Provisional application No. 60/813,525, filed on Jun. 14, 2006.

(51) Int. Cl. A61B 18/18

(52) U.S. Cl. 606/10; 606/41

(57) ABSTRACT

A surgical ablation system and method of treatment for creating lesions in tissue, including cardiac tissue for the treatment of arrhythmias and other diseases are disclosed. The ablation system includes a chest wall platform, introducer sheath, and ablation device. The system provides a stable platform for entering the heart while accommodating a beating heart. The method can include the steps of accessing a heart via a thoracic incision, deploying an ablation instrument within the heart and activating the ablation instrument to create at least one conduction-blocking lesion.

### CLAIM OF PRIORITY UNDER 35 U.S.C. §119

The present Application for Patent claims priority to Provisional Application No. 60/813,525 filed Jun. 14, 2006 and assigned to the assignee hereof and hereby expressly incorporated by reference herein.

### BACKGROUND OF THE INVENTION

The present invention relates to cardiac surgery and, in particular, surgical treatment of atrial fibrillation on a beating heart.

Cardiac arrhythmias, e.g., fibrillation, are irregularities in the normal beating pattern of the heart and can originate in either the atria or the ventricles. For example, atrial fibrillation is a form of arrhythmia characterized by rapid randomized contractions of the atrial myocardium. The regular pumping function of the heart is replaced by a disorganized, ineffective quivering as a result of chaotic conduction of electrical signals through the upper chambers of the heart. Atrial fibrillation is often associated with other forms of cardiovascular disease, including congestive heart failure, rheumatic heart disease, coronary artery disease, left ventricular hypertrophy, cardiomyopathy or hypertension.

Various surgical techniques have been proposed for the treatment of arrhythmia. Although these procedures were originally performed with a scalpel, these techniques may also use ablation (also referred to as coagulation) wherein the tissue is treated, generally with heat or cold, to cause tissue necrosis (i.e., cell destruction). The destroyed muscle cells are replaced with scar tissue which cannot conduct normal electrical activity within the heart.

The region of the pulmonary veins has been identified as one of the origins of errant electrical signals responsible for triggering atrial fibrillation. In one known approach, circumferential ablation of tissue within the pulmonary veins or at the ostia of such veins has been practiced to treat atrial fibrillation. Similarly, ablation of the region surrounding the pulmonary veins as a group has also been proposed. By ablating the heart tissue (typically in the form of linear or curved lesions) at selected locations, electrical conductivity from one segment to another can be blocked and the resulting segments become too small to sustain the fibrillatory process on their own. Ablation procedures are often performed during coronary artery bypass and mitral valve replacement operations because of a heightened risk of arrhythmias in such patients and the opportunity that such surgery presents for direct access to the heart.

Several types of ablation devices have been proposed for creating lesions to treat cardiac arrhythmias, including devices which employ electrical current (e.g., radio-frequency “RF”), heating or cryogenic cooling. Such ablation devices have been proposed to create elongated lesions that extend through a sufficient thickness of the myocardium to block electrical conduction.

These devices, however, are not without their drawbacks. The amount of time necessary to form a lesion is a critical factor in cardiac surgery. Because these devices rely upon resistive and conductive heating (or cooling), they typically must be placed in direct contact with the heart and such contact must be maintained for a considerable period of time to form a lesion that extends through the entire thickness of the heart muscle. The total length of time to form the necessary lesions can be excessive. This is particularly problematic for procedures that are performed upon a “beating heart” patient. In such cases, the heart itself continues to beat and is filled with blood, thus providing a heat sink (or reservoir) that works against conductive and/or resistive ablation devices. As “beating heart” procedures become more commonplace (in order to avoid the problems associated with arresting a patient's heart and placing the patient on a pump), the need for better ablation devices will continue to grow.

Ablation devices that employ radiant energy have also been proposed. These devices achieve rapid and effective photoablation through diffuse infrared radiation. Ablation devices that employ radiant energy create lesions in less time and with less risk of the adverse types of tissue destruction commonly associated with other types of ablation devices. Unlike instruments that rely on thermal conduction or resistive heating, controlled penetrating radiant energy can be used to simultaneously deposit energy throughout the full thickness of a target tissue, such as a heart wall, even when the heart is filled with blood. Distributed radiant energy also produces better defined and more uniform lesions.

While radiant energy ablation devices can efficiently produce uniform lesions, the existing instruments suffer from various design limitations. For example, instruments that are flexible enough to accommodate a beating heart may not provide sufficient stability to perform ablation with the desired precision.

Accordingly, there exists a need for better surgical ablation system that can efficiently form lesions while accommodating a beating heart.

### SUMMARY OF THE INVENTION

A surgical ablation system and treatment method is disclosed for creating lesions in tissue, especially cardiac tissue for treatment of arrhythmias and the like. The system is especially useful in port access cardiac surgery for accurate and efficient creation of lesions while accommodating a beating heart. The system can be applied to form endocardial ablations and is designed to create lesions in the atrial tissue in order to electrically decouple tissue segments on opposite sides of the lesion.

In one aspect of the invention, a system for performing endocardial ablation on a beating heart that includes a chest wall platform, introducer sheath, and ablation device is disclosed. It has been discovered that the accuracy and effectiveness of an ablation procedure on a beating heart can be increased if the surgeon is provided with a stable platform for entering the heart. The system includes a chest wall platform adapted to surround at least a portion of a beating heart and provide a stable staging area for entering the heart. In one preferred embodiment, the chest wall platform is adapted to stabilize and/or assist in aiming or directing at least one tool during the procedure.

The system also includes an introducer sheath couplable to the platform and capable of penetrating the heart wall to provide access to the endocardium. In one embodiment, the introducer sheath includes a hemostatic septum for preventing air entry into and/or blood leakage out of the left atrium. The introducer sheath can further include a trocar that is sufficiently flexible to accommodate a beating heart but capable of being stable at the working port. In another embodiment, the trocar can be adapted to seal against the left atrial appendage to inhibit leakage.

In another aspect of the invention, the introducer sheath can have electrical contacts positioned circumferentially around the catheter to ensure contact between the ablation device and the endocardium and/or to measure the effectiveness of a conduction block created by ablation of targeted tissue. In another embodiment, the system can further include a shuttle having electrical contacts formed thereon and adapted to slidably move along the sheath. Fiber optics can also be provided to measure conduction block.

The system also includes an ablation device adapted to pass through the introducer sheath and into the heart to ablate a target region of tissue. In one embodiment, the ablation device can be adapted to pass over an anchoring catheter. Alternatively, in another embodiment, the ablation device can be adapted to pass within a balloon catheter. The ablation device can further comprise one or more optical fibers and a lens, a reflector, or other optics adapted to direct light toward a target region of tissue.

The present invention also provides methods for ablating tissue. Generally, a method of treating atrial fibrillation on a beating heart is provided. The method includes accessing a beating heart via a thoracic incision and opening an entry site into the heart. A stable platform surrounding at least a portion of the beating heart can be established, and an introducer sheath can be passed into the heart via the platform. An ablation device can be inserted through the introducer sheath and deployed near a target region of tissue. The method further comprises activating the ablation device to form at least one lesion to block electrical conduction associated with fibrillation.

In another aspect of the method, the ablation device is introduced in conjunction with a balloon catheter for anchoring the device. In one embodiment, the ablation device is passed over the balloon catheter. In another embodiment, the ablation device is passed within the balloon catheter.

The method can further comprise verifying that the catheter is in contact with the endocardium. Additionally, the method can include flushing the target region of tissue to keep it free from blood. The method can optionally include verifying the creation of a conduction block.

### BRIEF DESCRIPTION OF THE DRAWINGS

The invention will be more fully understood from the following detailed description taken in conjunction with the accompanying drawings, in which like reference numerals designate like parts throughout the figures, and wherein:

FIG. 1 is a schematic perspective view of a chest wall platform according to the invention as applied to a beating heart;

FIG. 1A is a schematic illustration of a human torso, showing the entry ports that can be accessed via the chest wall platform of FIG. 1;

FIG. 2 is a schematic perspective view of a human heart illustrating an access method according to the invention;

FIG. 2A is a side perspective view of an introducer sheath;

FIG. 3 is a perspective view of a human heart illustrating a further step in accessing the heart;

FIG. 3A is a side perspective view of a needle and guidewire assembly;

FIG. 4A is a side view of an ablation device adapted to pass over a balloon catheter;

FIG. 4B is a side view of an ablation device adapted to pass within a balloon catheter;

FIG. 5 is a schematic view of an endocardial ablation system as inserted into the left atrium;

FIG. 5A is a side perspective view of the ablation device of FIG. 5;

FIG. 6 is a side view of another embodiment of an ablation device anchored in a pulmonary vein;

FIG. 7 is a schematic perspective view of another endocardial ablation system according to the invention illustrating the formation of the encircling lesions;

FIG. 8 is a schematic perspective view of yet another embodiment of an ablation tool useful in creating a connecting lesion to the mitral valve annulus;

FIG. 9 is a schematic view of the ablation tool of FIG. 8 used to create the connecting lesion to the mitral valve annulus;

FIG. 10 is a perspective view of a human heart with further surgical tools useful in practicing the invention (a bulldog-style clip and applier) positioned at the left atrial appendage;

FIG. 11 is a schematic illustration of a method according to the invention for closure of the left atrial appendage;

FIG. 11A is a further schematic illustration of a staple closure method;

FIG. 11B is a further schematic illustration of a suture closure method; and

FIG. 11C is a further schematic illustration of an elastic closure method.

### DETAILED DESCRIPTION OF INVENTION

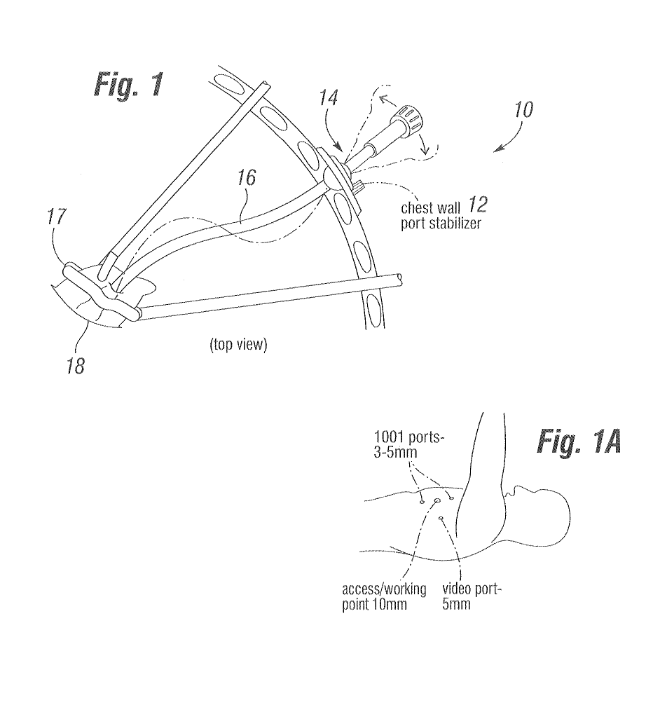

The present invention provides a system and method for minimally invasively treating atrial fibrillation on a beating heart using an endocardial approach. As shown in FIGS. 1-4, the endocardial ablation system 10 generally includes a chest wall platform 12, an introducer sheath 20 couplable to the platform 12, and an ablation device 40 adapted to pass through the introducer sheath 20. In use, the system can be applied endocardially to ablate a target region of tissue.

The chest wall platform 12 of the ablation system 10, shown in FIG. 1, is adapted to surround at least a portion of a beating heart 18 and provide a stable platform for entering the heart 18. The configuration of the platform will vary with the mode of access. For example, if the heart 18 is accessed via thorascopic access ports, the platform 12 is adapted to mate to a working port 14. If the heart 18 is accessed via a left side mini-thoracotomy incision, the platform 12 is adapted to mate to a retractor. The platform 12 can be adapted to stabilize and/or assist in aiming or directing at least one tool during the procedure.

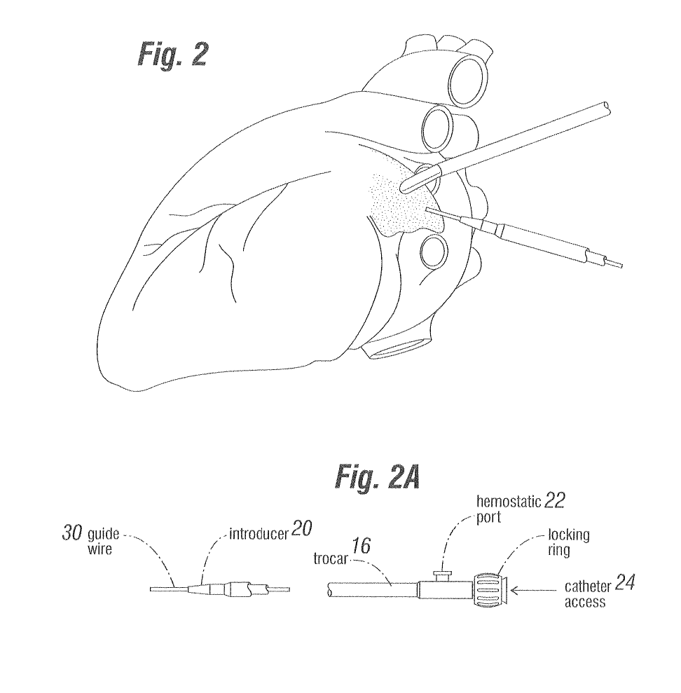

The introducer sheath 20 is shown in FIG. 2. The sheath can be coupled to the platform 12 and is capable of penetrating the heart wall to provide access to the endocardium. One purpose of the sheath 20 is to protect the left atrial appendage (LAA) during tool insertion and removal. The introducer sheath 20 can be configured to prevent air entry in and blood leakage out. One embodiment of this concept includes providing the introducer sheath 20 with a hemostatic septum 22. The introducer sheath 20 can also be configured to limit tension or straining on the LAA. For example, the introducer sheath 20 can be provided with a flexible trocar 16 adapted to move with the beating heart 18 yet remain stable at the working port 14. The introducer sheath 20 can further include an interface to a second facilitation tool or subassembly that allows the trocar 16 to seal against the LAA to inhibit leakage. One embodiment of this concept is a feature on the trocar 16 that allows deployment of a band, suture, or other means of tightening to seal the trocar 16 against the LAA. Another embodiment of this concept includes providing the introducer sheath 20 with a clamping tool 17 capable of axially securing the introducer sheath 20 and reducing bleeding from the wound in the LAA. In addition to inhibiting leakage, the clamp 17 also helps to stabilize the LAA.

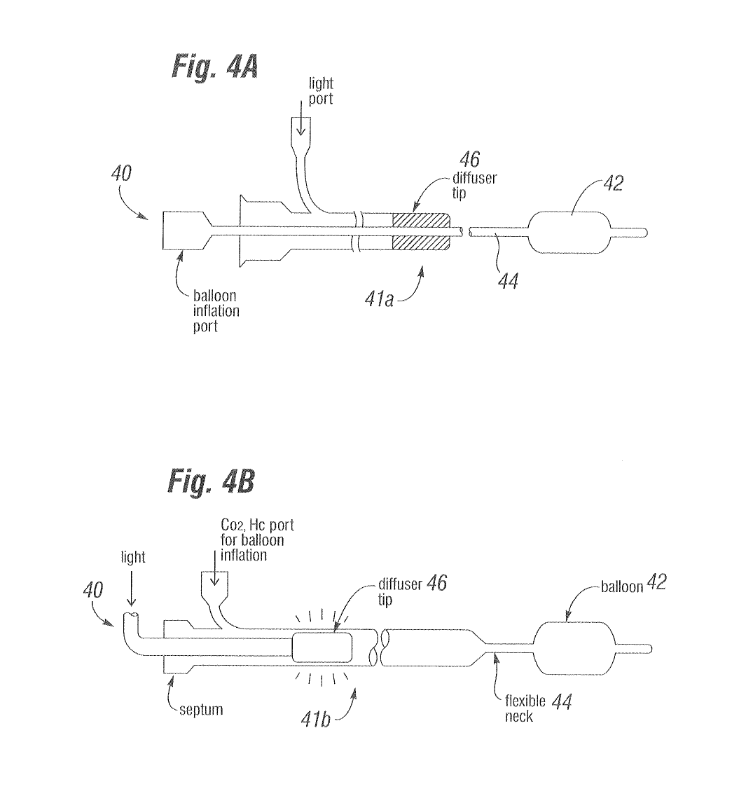

The ablation device 40, shown in FIGS. 4A-4B, is adapted to pass through the introducer sheath 20 and is deployable in the heart to ablate a target region of tissue. The ablation device 40 can be introduced into the heart in conjunction with a balloon catheter that is used for anchoring. In one embodiment, shown in FIG. 4A, the ablation device 40 is adapted to pass over a balloon catheter 41a. In another embodiment, shown in FIG. 4B, the ablation device 40 is adapted to pass within a balloon catheter 41b. In either embodiment, the balloon catheter can include the following features. The balloon catheter can be a sausage type balloon 42 for pulmonary vein anchoring. The system can include multiple balloon sizes 52 to accommodate varying pulmonary vein diameters. A flexible neck 44 can be provided between the balloon 42 and the distal end of the ablation catheter sheath to allow for easy orientation and placement of the catheter against the endocardium once the balloon has been deployed.

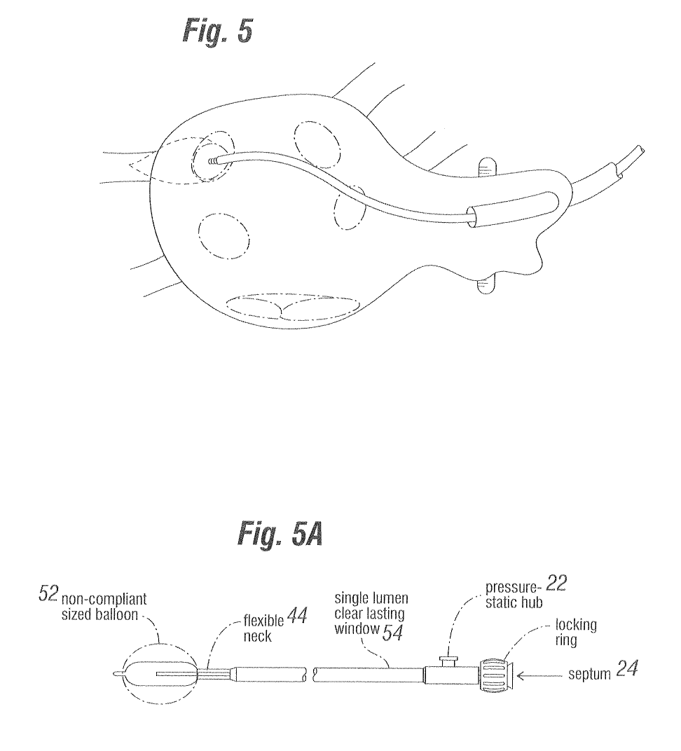

As shown in FIG. 5, the ablation catheter can also include an optically clear ablation catheter wall 54 to allow for light transmission at the appropriate wavelength to create ablation. In this embodiment, the energy antenna or diffuser tip 46 is pre-assembled in the ablation catheter sheath. The ablation device 40 can further include an irrigation system (not shown) to allow flushing of the ablation area to keep it free of blood. A reflector (not shown) adapted to direct light toward a target region of tissue can also be provided.

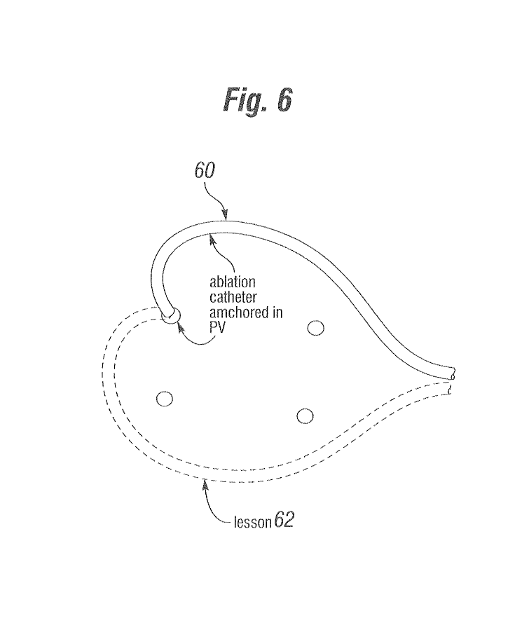

As shown in FIG. 6, the ablation device 40 can optionally include a pre-formed loop 60 on the ablation catheter sheath to allow for the creation of a catheter loop against the endocardium thereby enabling the creation of an encircling lesion 62. Multiple loop sizes can be provided to accommodate different sized atria.

Additionally, the ablation device can include detection elements (not shown) configured to ensure contact between the ablation device and the endocardium and/or to measure the conduction block in the targeted tissue. In one embodiment, electrical contacts can be positioned circumferentially around the catheter to detect whether the catheter is in contact with the endocardium and/or to verify the conduction block. In another embodiment, the system 10 further includes a shuttle (not shown) configured to ride along the ablation catheter. The shuttle includes electrical contacts for detecting contact with the endocardium and/or verifying the conduction block. In a third embodiment, the conduction block can be verified using fiber optics.

In use, the endocardial ablation system 10 is used for minimally invasively treating artrial fibrillation on a beating heart. The treatment involves entering the left atrium (LA) via several possible access points and creating one or more transmural myocardial lesions via ablation. Various entry approaches into the LA have been identified and include entering through the LAA, an incision or puncture through the LA wall, through a pulmonary vein, and through a transseptal percutaneous femoral vein approach. For illustration purposes, this description assumes entry through the LAA, however the embodiments of the endocardial ablation procedure described herein apply to any LA entrance approach. The ablation procedure generally comprises accessing the endocardial surfaces of the LA, introducing an ablation device into the LA, positioning the device adjacent the endocardium, and delivering energy to the myocardium to create sections of damaged tissue that block electrical conduction.

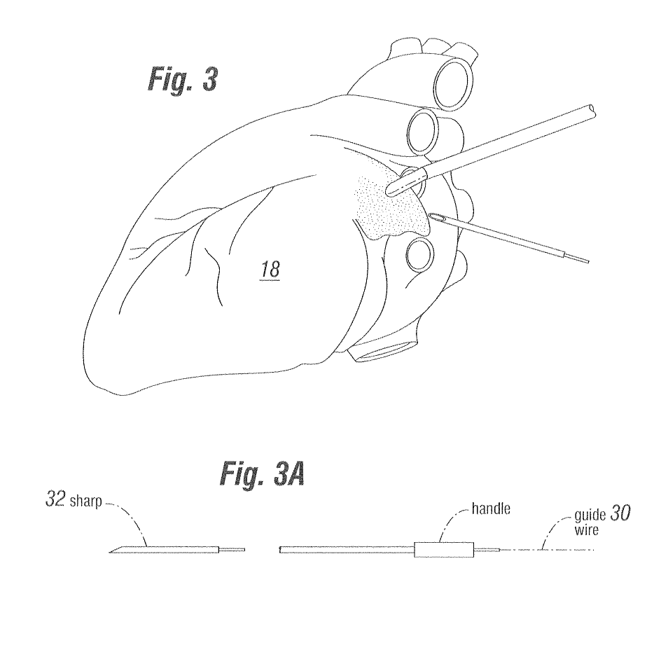

The endocardial surfaces of the LA can be accessed via thorascopic access ports or a left side mini-thoracotomy incision. In the case of a mini-thoracotomy, a retractor is placed. In the case of a thoracoscopic approach, working ports are inserted. After the retractor or working port is inserted, the chest wall platform 12 is secured. The pericardium may be suspended to improve access to the LAA. An incision is made in pericardium local to the LAA area such that the LAA is exposed. Using graspers, the LAA is pulled towards the surgical opening in the patient. An appropriate incision area is identified in the LAA. This incision area is likely near the tip of the appendage, as far away from the base as possible, to allow room for ligation, excising, or ablating near or around the base. As shown in FIGS. 3 and 3A, a guidewire 30 is inserted into the LA through a custom needle 32. The needle can be removed, and confirmation of the location of the guidewire 30 can be made either via transesophageal echocardiogram (TEE), fluoroscopy, or other visualization means.

As shown in FIGS. 2 and 2A, a customized introducer sheath 20 can be inserted over the guidewire 30 and into the LA. Once the introducer sheath 20 is in place, the inner piece of the introducer assembly is removed and blood is aspirated to ensure patency. Fool access into the LA for ablation is now possible by passing an ablation catheter through a septum 24 on the proximal end of the sheath 20.

The ablation process described in this invention consists of a series of steps that can include tool positioning, tool anchoring, ablation, and verification of conduction block. During tool positioning, the guidewire 30 tip can be inserted into either the inferior or superior right pulmonary veins. The positioning of the guidewire 30 can be visualized with TEE or fluoroscopy. Once the guidewire 30 is located in a right pulmonary vein, a pre-flushed customized balloon catheter 41a, 41b can be inserted over the guidewire 30. The balloon 42 is inflated to anchor the catheter in the pulmonary vein for ablation.

Once the catheter system is anchored via the balloon catheter 41a, 41b, the diffusion tip 46 is advanced. The flexible neck 44 and pre-formed shape allow it to be correctly positioned against the endocardium. The ablation procedure can optionally include the step of ensuring contact with the endocardium via electrical contacts positioned circumferentially around the catheter or catheter shuttle. Once the ablation device 40 is properly positioned, the energy antenna or diffusion tip 46 call be activated to ablate. In one embodiment, the ablation procedure further includes flushing the ablation area to keep it free of blood. After activation, the conduction block can be verified via electrical contacts positioned on the catheter or catheter shuttle. In another embodiment, the conduction block can be verified with fiber optics. Once conduction block has been achieved, the diffusion tip 46 can be moved to the next position and the process repeated.

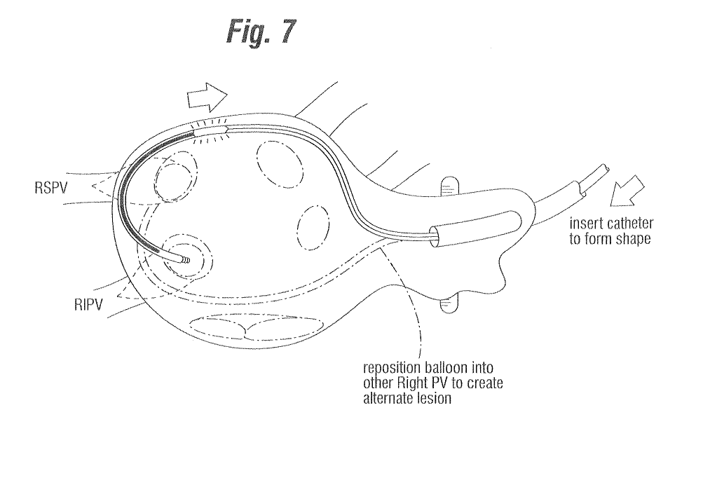

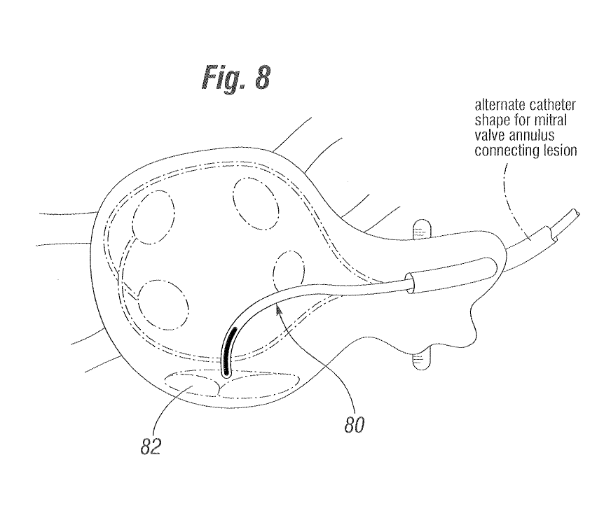

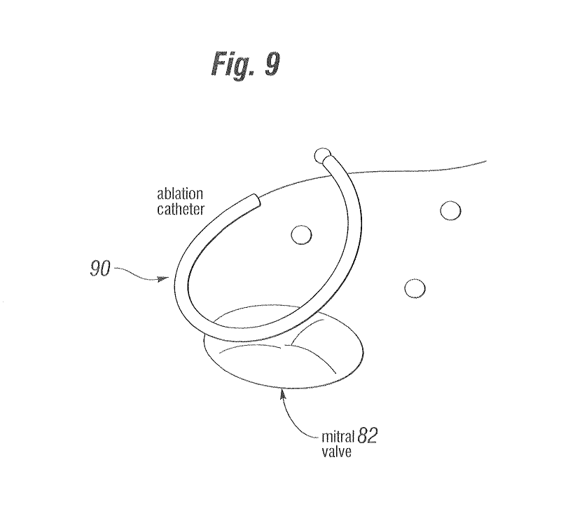

For complete encirclement around all four pulmonary veins, the catheter can be repositioned on the other side of the LA creating a closed circle lesion. To ensure continuity between the two lesions, the ablation catheter can be repositioned in the alternate right pulmonary vein as shown in FIG. 7. This set of lesions creates the pulmonary vein encircling lesion and the LAA connecting lesion of the Cox-Maze lesion set simultaneously. As shown in FIG. 8, a separate ablation tool 80 may be needed to create the connecting lesion to the mitral valve 82 annulus. For example, FIG. 9 shows that a similar tool 90 with a different preformed loop can be used.

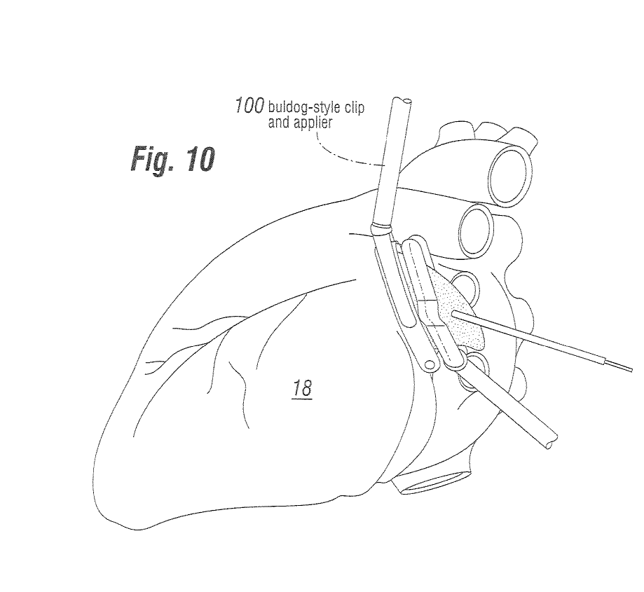

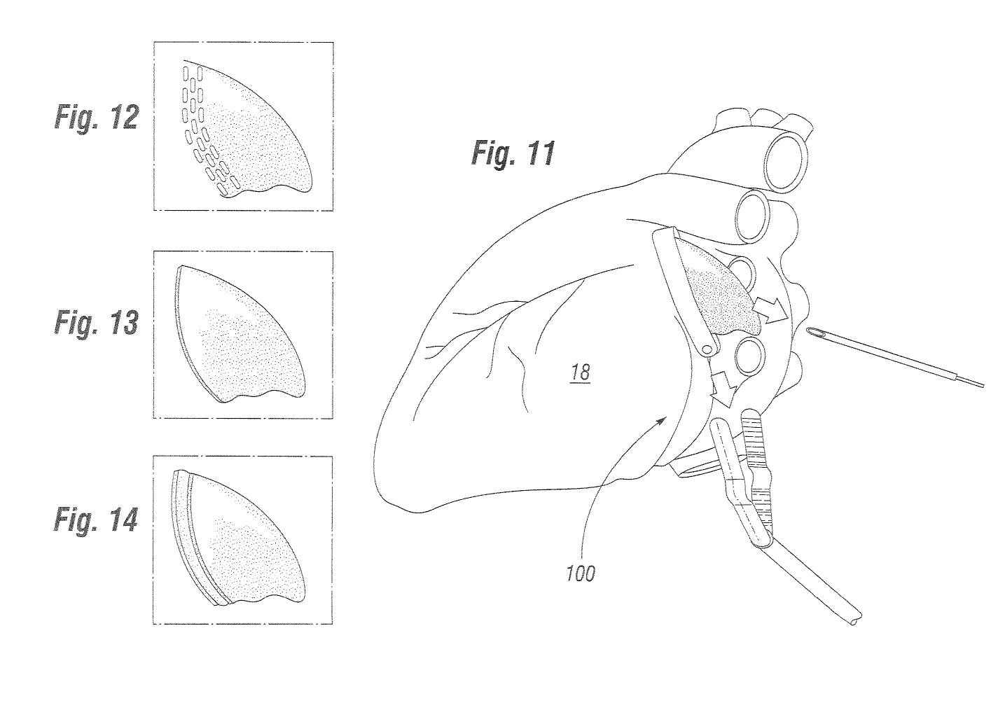

Once the ablations are complete, the tools can be removed from the LAA and the opening in the LAA closed. Possible closure methods include ligation, stapling, suturing, rubber bands, or surgical clip. FIGS. 10 and 11 illustrate closure via a bulldog-style clip and applier 100 as well as several alternate closure methods. In one embodiment, closure of the LAA includes excluding it from circulation but not excising it from the heart. In another embodiment, closure of the LAA includes excluding it from circulation by one of several means including but not limited to stapling, suturing, occluding and excising it from the heart.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method of treating atrial fibrillation on a beating heart comprising:
accessing a beating heart via a thoracic incision,
opening an entry site into the heart,
establishing a stable platform surrounding at least a portion of the beating heart,
passing an introducer sheath into the heart via the platform,
inserting an ablation device through the introducer sheath,
deploying the ablation device near a target region of tissue, and
activating the ablation device to form at least one lesion to block electrical conduction associated with fibrillation.

2. The method of claim 1, wherein the step of accessing the heart further comprises accessing the heart under laproscopic guidance.

3. The method of claim 1, wherein the step of accessing the heart further comprises accessing the left atrium through the left atrial appendage.

4. The method of claim 1, wherein the step of accessing the heart further comprises creating at least one thorascopic access port.

5. The method of claim 4, wherein the step of accessing the heart further comprises inserting working ports to provide access to the left atrial appendage.

6. The method of claim 1, wherein the step of accessing the heart further comprises a left side mini-thoracotomy incision.

7. The method of claim 6, wherein the step of accessing the heart further comprises using a retractor to provide access to the left atrial appendage.

8. The method of claim 1, wherein the step of accessing the heart further comprises pulling the left atrial appendage towards the surgical opening in the patient.

9. The method of claim 1, wherein the step of opening an entry site further comprises inserting a needle into the left atrium.

10. The method of claim 9, wherein the step of opening an entry site further comprises inserting a guidewire through a lumen of the needle.

11. The method of claim 1, wherein the step of passing an introducer sheath into the heart further comprises inserting the introducer sheath into the left atrium.

12. The method of claim 10, wherein the step of passing an introducer sheath into the heart further comprises inserting the guidewire tip into either the inferior or superior right pulmonary veins.

13. The method of claim 10, wherein the step of passing an introducer sheath into the heart further comprises inserting a balloon catheter over the guidewire.

14. The method of claim 13, wherein the step of passing an introducer sheath into the heart further comprises inflating the balloon to anchor the catheter.

15. The method of claim 1, wherein the step of inserting an ablation device through the introducer sheath further comprises introducing the ablation device in conjunction with a balloon catheter.

16. The method of claim 1, wherein the step of inserting an ablation device through the introducer sheath further comprises introducing the ablation device over the balloon catheter.

17. The method of claim 1, wherein the step of inserting an ablation device through the introducer sheath further comprises introducing the ablation device within a balloon catheter.

18. The method of claim 1, wherein the step of deploying the ablation device further comprises positioning the ablation device against the endocardium.

19. The method of claim 1, wherein the step of deploying the ablation device further comprises verifying that the catheter is in contact with the endocardium.

20. The method of claim 1, wherein the step of activating the ablation device is repeated after repositioning the catheter to another location in the left atrium.

21. The method of claim 1, wherein the step of activating the ablation device further comprises flushing the target region of tissue to keep it free from blood.

22. The method of claim 1, wherein the step of activating the ablation device further comprises verifying the creation of a conduction block.

23. The method of claim 1, wherein the left atrial appendage is closed and excluded from circulation but not excised from the heart.

24. The method of claim 1, wherein the left atrial appendage is closed and excluded from circulation by stapling, suturing, occluding, or excising it from the heart.

25. A system for performing endocardial ablation on a beating heart comprising:
a chest wall platform adapted to surround at least a portion of a beating heart and provide a stable platform for entering the heart,
an introducer sheath couplable to the platform capable of penetrating the heart wall and providing access to the endocardium, and
an ablation device adapted to pass through the introducer sheath and deployable in the heart to ablate a target region of tissue.

26. The system of claim 25, wherein the chest wall platform is adapted to stabilize at least one tool during the procedure.

27. The system of claim 25, wherein the chest wall platform is adapted to assist ill aiming or directing at least one tool during the procedure.

28. The system of claim 25, wherein the introducer sheath is configured to prevent air entry into the left atrium.

29. The system of claim 25, wherein the introducer sheath is configured to prevent blood leakage out of the left atrium.

30. The system of claim 25, wherein the introducer sheath includes a hemostatic septum.

31. The system of claim 25, wherein the introducer sheath is adapted to limit tension or straining on the left atrial appendage.

32. The system of claim 25, wherein the introducer sheath further includes a trocar that is sufficiently flexible to accommodate a beating heart but capable of being stable at the working port.

33. The system of claim 32, wherein the trocar is adapted to seal against the left atrial appendage to inhibit leakage.

34. The system of claim 33, wherein the trocar is adapted to deploy a band, suture, or other means of tightening to seal the trocar against the left atrial appendage.

35. The system of claim 33, wherein the system includes a clamping tool adapted to secure the introducer sheath axially.

36. The system of claim 25, wherein the system further includes electrical contacts positioned circumferentially around the catheter.

37. The system of claim 25, wherein the introducer sheath further includes a shuttle having electrical contacts formed thereon and adapted to slidably move along the sheath.

38. The system of claim 25, wherein the system further includes fiber optics configured to measure conduction block.

39. The system of claim 25, wherein the ablation device is adapted to pass over a balloon catheter that is used for anchoring.

40. The system of claim 25, wherein the ablation device is adapted to pass within a balloon catheter that is used for anchoring.

41. The system of claim 25, wherein the ablation device further includes a reflector adapted to direct light toward a target region of tissue.
